(19) United States
(12) Patent Application Publication
(10) Pub. No.: US 2023/0257829 A1
(43) Pub. Date: Aug. 17, 2023

(54) DNA-METHYLATION-BASED QUALITY CONTROL OF THE ORIGIN OF ORGANISMS

(71) Applicants: Evonik Operations GmbH, Essen (DE); DEUTSCHES KREBSFORSCHUNGSZENTRUM STIFTUNG DES ÖFFENTLICHEN RECHTS, Heidelberg (DE)

(72) Inventors: Sina TÖNGES, Heidelberg (DE); Frank LYKO, Heidelberg (DE); Geetha VENKATESH, Heidelberg (DE); Ranja ANDRIANTSOA, Heidelberg (DE); Fanny GATZMANN, Heidelberg (DE); Florian BÖHL, Neckargemünd (DE); Andreas KAPPEL, Glashütten (DE); Emeka Ignatius IGWE, München (DE); Frank THIEMANN, Nottuln (DE)

(73) Assignees: Evonik Operations GmbH, Essen (DE); DEUTSCHES KREBSFORSCHUNGSZENTRUM STIFTUNG DES ÖFFENTLICHEN RECHTS, Heidelberg (DE)

(21) Appl. No.: 18/018,398

(22) PCT Filed: Jul. 23, 2021
(86) PCT No.: PCT/EP2021/070683

§ 371 Date: Jan. 27, 2023

(30) Foreign Application Priority Data
Jul. 30, 2020 (EP) 20188761.9

(51) Int. Cl. C12Q 1/6888; C12Q 1/6869

(52) U.S. Cl. CPC C12Q 1/6888; C12Q 1/6869; C12Q 2600/154; C12Q 2600/166

(57) ABSTRACT

The invention pertains to a method for the identification of the geographic origin of an individual test subject or of an individual group of test subjects, the method comprising the comparison of a test methylation profile obtained from genomic material of the individual test subject or of the individual group of test subjects with one or more predetermined reference methylation profiles each being specific for a distinct geographic origin.

### FIELD OF THE INVENTION

The invention is based on the finding that specific panels of genes provide a source for the generation of DNA methylation profiles which are specific for a geographic origin of organisms. In particular, DNA methylation profiling may be used to identify the genetic origins of animals, that include rearing animals also known as livestock, such as crabs, fish or chicken. The methods of the invention can be applied to identify the geographic origin of organisms including rearing animals, to control assumed geographic origins of a sample of the organisms including rearing animals, and for assessing environmental parameters of habitats of organisms including rearing animals. Further, the invention provides quality control methods and processes for developing new test systems for various organisms including rearing animals.

### BACKGROUND OF THE INVENTION

Sustainable food production is presently considered among the globally most important societal needs. As the value chains of the agriculture and aquaculture industries are highly complex, certificates have been established to reinforce consumer relationships and trust. However, certificates are based on audits at specific farms and can be easily tampered by moving livestock from non-certified farms to certified farms. Furthermore, surveillance of sustainable farming practices is spotty and largely limited to audits. As “bad” farming practices are widespread in the industry, there is an urgent need for a tampering-resistant certificate.

The livestock and food process industries have been heavily involved in developing strategies of identifying, tracing and managing the risks in the area of food safety, and in developing strategies for consumer information (transparent value chains). Health, safety and also animal welfare considerations demand that the origins of animal products, and in particular meat products, should be traceable, so that quality assurance audits, and monitoring procedures can be effectively and reliably carried out.

A comparison of genome-wide patterns of methylation and variation at the DNA level revealed that a highly significant proportion of epigenetic variation could be associated with fitness differences and rearing conditions such as captivity in salmon (Le Luyer J et al. 2017 PNAS vol 114, no 49).

A study of genome wide methylation in the marbled crayfish (Procambarus virginalis) observed stable methylation of most parts of the genome between animals and tissues while a subset of about 700 genes were demonstrated to be highly variable in their methylation (Gatzmann, F. DNA methylation in the marbled crayfish Procambarus virginalis. PhD thesis, Faculty of Biosciences, University of Heidelberg, 2018).

In view of the above, there is an urgent need to provide means for identifying and quality controlling the geographic origin of organisms, in particular food and more particularly animal material derived from rearing stock.

### SUMMARY OF THE INVENTION

The aforementioned objective is solved by the different aspects of the present invention. The invention is based on the finding that resilience to environmental exposures such as stress, climate, light or diet is a fundamental concept of biology and results in the adaptation of an organism to its environment. The capability to adapt to the environment and maintain the adapted biological pattern depends on epigenetic mechanisms, including DNA methylation.

The inventors have unexpectedly found that this property can be utilized to identify environment-specific “epigenetic fingerprints” on the genome and to align organisms to the ecosystem they are originating from. Based on these findings, the present invention provides methods to identify the geographic origin of organisms including rearing animals also known as livestock, methods to control assumed geographic origins of a sample of organisms including rearing animals, and methods for assessing environmental parameters of habitats of organisms including rearing animals. Further, the invention provides quality control methods and processes for developing new test systems for various organisms including rearing animals

Generally, and by way of brief description, the main aspects of the present invention can be described as follows:

In a first aspect, the invention pertains to a method for the identification of the geographic origin of an individual test subject or of an individual group of test subjects, the method comprising the comparison of a test methylation profile obtained from genomic material of the individual test subject or of the individual group of test subjects with one or more predetermined reference methylation profile(s) each being specific for a distinct geographic origin.

In a second aspect, the invention pertains to a method for quality controlling a suspected geographic origin of an individual test subject or individual group of test subjects, the method comprising the steps of
a. determining the methylation status of one or more pre-selected methylation sites within genomic material contained in a biological sample obtained from the individual test subject, or of the individual group of test subjects;
b. determining from the methylation status determined in (a) a test methylation profile of the individual test subject, or of the individual group of test subjects; and
c. comparing the test methylation profile determined in (b) with a predetermined reference methylation profile, wherein the predetermined reference methylation profile is specific for individual subjects, or individual groups of subjects, of the same biological taxon (preferably species) of the individual test subject or of the individual group of test subjects, and which were obtained from the suspected geographic origin;
wherein if the test methylation profile is significantly similar to the predetermined reference methylation profile, the individual test subject or individual group of test subjects passes the quality control and the suspected geographical origin is indicated as true geographical origin.

In a third aspect, the invention pertains to a method for assessing one or more environmental parameters of a habitat of an individual test subject, or of an individual group of test subjects, the method comprising the steps of
(a) determining the methylation status of one or more pre-selected methylation sites within the genomic material contained in a biological sample obtained from the individual test subject, or of the individual group of test subjects;
(b) determining from the methylation status determined in (a) a test methylation profile of the individual test subject, or individual group of test subjects; and
(c) comparing the test methylation profile determined in (b) with one or more predetermined reference methylation profiles, wherein the one or more predetermined reference methylation profiles are each specific for individual subjects, or individual groups of subjects, of the same biological taxon (preferably species) of the individual test subject or individual group of test subjects, and which were each obtained from distinct geographic origins; and wherein the distinct geographic origin is distinguished from other distinct geographic origins by one or more environmental parameters;
wherein if the test methylation profile is significantly similar to one of the one or more predetermined reference methylation profiles, the individual test subject or the individual group of test subjects is derived from a geographical origin having similar, or preferably equal, environmental parameters to the geographical origin of the subjects or group of subjects of the one of the one or more predetermined reference methylation profiles.

In a fourth aspect, the invention pertains to a method for confirming or declining an assumed geographic origin of an individual test subject or of an individual group of test subjects, the method comprising the comparison of a test methylation profile obtained from genomic material of the individual test subject or of the individual group of test subjects with one or more predetermined reference methylation profiles each being specific for a distinct geographic origin.

In a fifth aspect, the invention pertains to a method for developing a test system for confirming an assumed geographic origin of an individual test subject or of an individual group of test subjects, the method comprising the steps of:
(a) determining the methylation status of one or more methylation sites within genomic material contained in a biological sample obtained from the individual test subject, or of the individual group of test subjects;
(b) selecting from the one or more methylation sites a reference panel of methylation sites which is characterized by a specific and distinct differential methylation profile for each of the known geographic origins;
(c) obtaining a test system by assigning a reference methylation profile for each of the known geographic origins (or locations); and
wherein a comparison of a test methylation profile obtained from a test sample with the reference methylation profiles obtained in (c) allows for confirming the assumed geographic origin of the individual test subject from which the test sample was obtained.

### DETAILED DESCRIPTION OF THE INVENTION

In the following, the elements of the invention will be described. These elements are listed with specific embodiments and/or examples; however, it should be understood that these elements may be combined in any manner and in any number to create additional embodiments and/or examples. The variously described examples and preferred embodiments should not be construed to limit the present invention to only the explicitly described embodiments or examples. This description should be understood to support and encompass embodiments and examples which combine two or more of the explicitly described embodiments or which combine the one or more of the explicitly described embodiments or examples with any number of the disclosed and/or preferred elements. Furthermore, any permutations and combinations of all described elements in this application should be considered disclosed by the description of the present application unless the context indicates otherwise.

The terms “of the present invention”, “in accordance with the present invention”, “according to the present invention” and the like, as used herein are intended to refer to all aspects, embodiments and examples of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that the application of the teachings according to any aspect of the present invention to a specific problem or environment, and the inclusion of variations according to any aspect of the present invention or additional features thereto (such as further aspects and embodiments or examples), will be within the capabilities of one having ordinary skill in the art in light of the teachings contained herein.

Unless context dictates otherwise, the descriptions and definitions of the features set out within this description are not limited to any particular aspect or embodiment of the invention and apply equally to all aspects and embodiments which are described.

All references, patents, and publications cited herein are hereby incorporated by reference in their entirety.

The term “geographic origin” in context of the herein defined invention shall pertain to a geographic location which is distinguished from other geographic locations by one or more environmental parameters of the subject or group of subjects. Such environmental parameters depend on the habitat of the subject or group of subjects and may be different in case the subject or group of subject lives or is cultured in water, on or in soil, or may be selected from a food or air parameter etc. As non-limiting examples of the present invention, for sweet water crabs (such as the marbled crayfish), environmental parameters may be selected from pH, water hardness, manganese content, iron content, and aluminum content - as mentioned these parameters although preferred shall be understood as non-limiting illustrative examples and may greatly vary depending on the taxon or species of the subject or group of subjects. As such, a habitat for the subject or group of subjects that live in water, these habitats can be selected from standing or flowing waters such as lakes, rivers, aqua farms, other pools or bodies of water or ponds. A geographic origin shall be understood to be the geographic location that is considered to be a habitat wherein the individual test subject, or individual group of test subjects, were spawned and/or cultured, or at least cultured for a significant time during their lifetime.

The term “test” used in conjunction with the term subject in the present disclosure refers to an entity or a living organism that is subjected to the method according to any aspect of the present invention and is the basis for an analysis application of the present invention. An “(individual) test subject”, an “(individual) group of test subjects” or a “test profile” is therefore a (individual) subject or group of subjects being tested according to the invention or a profile being obtained or generated in this context. Conversely, the term “reference” shall denote, mostly predetermined, entities which are used for a comparison with the test entity.

A subject or group of subjects in context of the present invention may be any living organism. For example, a subject according to any aspect of the present invention may be a plant or animal of any kind, preferably a rearing animal (or rearing stock) or livestock, which may be vertebrates or invertebrates. Typical examples of invertebrates that may be useful for being a subject according to any aspect of the present invention may be prawn or crabs such as the marbled crayfish. Typical examples of vertebrates that may be useful for being a subject according to any aspect of the present invention may be fish or land animals such as chicken or other livestock that may be cultured.

The term “genomic material” shall refer to nucleic acid molecules or fragments of the genome of the subject or group of subjects. Preferably such nucleic acid molecules or fragments are DNA or RNA or hybrids thereof, and most preferably are molecules of the DNA genome of a subject or group of subjects.

In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro.

As used herein, a “methylated nucleic acid molecule” refers to a nucleic acid molecule that contains one or more nucleotides that is/are methylated.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745).

The term “bisulfite” as used herein encompasses any suitable type of bisulfite, such as sodium bisulfite, or another chemical agent that is capable of chemically converting a cytosine (C) to a uracil (U) without chemically modifying a methylated cytosine and therefore can be used to differentially modify a DNA sequence based on the methylation status of the DNA, e.g., U.S. Pat. Pub. US 2010/0112595 (Menchen et al.). As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

In context of the present invention also any “non-bisulfite-based method” and “non-bisulfite-based quantitative method” are comprised to test for a methylation status at any given methylation site to be tested. Such terms refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

A “biological sample” in context of the invention may comprise any biological material obtained from the subject or group of subjects that contains genomic material, and may be liquid, solid or both, may be tissue or bone, or a body fluid such as blood, lymph, etc. In particular the biological sample useful for the present invention may comprise biological cells or fragments thereof.

As used herein, the term “pre-selected methylation sites” refers to methylation sites that were selected from genes or regions that showed the highest degree of methylation variation during the training of the method and fulfils certain quality criteria such as a minimum sequencing coverage of ≥5x were considered and for ≥5 qualified CpG sites. Additionally, genes that have an average methylation level <0.1 or an average methylation level >0.9 can be excluded due to their limited dynamic range. “Reference methylation profiles” may be defined on the basis of multiple training samples using multivariate statistical methods, such as such as Principal Component analysis or Multi-Dimensional Scaling.

The term “significantly similar” in context of the present disclosure, and in particular in context with the comparison of methylation profiles (such as the comparison between test profiles (from test subject(s) and reference profiles)) shall mean a similarity observed by statistical means (i.e. by using bioinformatics) and/or also by observation using the eye. A significant similarity is observed for example if a test profile overlaps with a reference profile that is defined by multiple training samples through multivariate statistical methods, such as Principal Component analysis or MultiDimensional Scaling. In particular, a test profile is significantly similar to the pre-determined reference profile if more than 50, 55, 60, 65, 70, 75, 80, 85, 90, 95% of the methylation pattern/profile overlaps with that of the reference profile. A similarity of a test profile to more than one, such as two, three or even all reference profile reduces the significance of the similarity.

The term “pre-determined reference profile” used in the context of the present invention refers to a typical or standard methylation profile of the genomic material of a living organism with a specific geographical origin. The pre-determined reference profile may be obtained from a control subject. For example, the control subject may a living organism of the same species as the test subject which has a known geographical origin. Alternatively, the pre-determined reference profile may be obtained from a variety of organisms living in the specific geographical origin. The methylation profile of different organisms of a specific geographical origin may be identical. There may be a compilation of several pre-determined reference profiles and comparing the methylation profile of the test subject with the pre-determined reference profiles in the compilation may enable identifying the specific pre-determined reference profile that is similar to the methylation profile of the test subject and then the geographical origin of the test subject may be deduced to be that of the predetermined reference profile.

The term “similar” used in relation to the geographical origin refers to the habitat or geographical origin of the test subject (s) based on the habitat or geographical origin of the organism from which the pre-determined reference profile was obtained. The term ‘similar’ may refer to the type of habitat, the environmental parameters of the habitat, the country where the habitat is located and the like. The geographical origin of the test subject may be 50, 55, 60, 65, 70, 75, 80, 85, 90, 95% similar to that of the geographical origin of the pre-determined reference profile based on at least one or more environmental parameters as defined above under ‘geographical origin’.

In a first aspect, the invention pertains to a method for the identification of the geographic origin of an individual test subject or of an individual group of test subjects, the method comprising the comparison of a test methylation profile obtained from genomic material of the individual test subject or of the individual group of test subjects with one or more predetermined reference methylation profiles each being specific for a distinct geographic origin.

The present invention is predicated on the surprising identification of methylation profiles in a subset of genes of living organisms including animals which are within one species characteristic for a distinct geographic origin of an individual of said species. Other individuals of the species which originate from a different geographic location are distinguishable by a different methylation profile for the same subset of genes - or methylation sites therein.

In one example of any aspect of the present invention, the method may preferably comprise the following method steps:
(a) determining the methylation status of one or more pre-selected methylation sites within the genomic material contained in a biological sample obtained from the individual test subject, or of the individual group of test subjects;
(b) determining from the methylation status determined in (a) a test methylation profile of the individual test subject, or of the individual group of test subjects; and
(c) comparing the test methylation profile determined in (b) with one or more predetermined reference methylation profiles, wherein each of the one or more predetermined reference methylation profiles is specific for a distinct geographic origin of subjects or group of subjects which are of the same biological taxon of the individual test subject or individual group of test subjects;
wherein if the test methylation profile is significantly similar to one of the one or more predetermined reference methylation profiles, the individual test subject or the individual group of test subjects has a geographical origin similar to the subjects or group of subjects of the one or more predetermined reference methylation profiles.

The individual test subject or individual group of test subjects may be any biological entity having a DNA genome and DNA genome methylation. Preferably the methylation site is a CpG site. The individual test subject or individual group of test subjects may be selected from a prokaryote, or a eukaryote, such as a unicellular or multicellular plant, a fungus or an animal.

In one aspect of the invention, the one or more pre-selected methylation sites in (a) are methylation sites associated with tissue specific gene expression. Preferably, the pre-selected methylation sites are associated with gene expression of one distinct tissue.

The tissue may be selected from
(i) metabolic tissue such as gut tissue, said gut tissue preferably being ileum or jejunum,
(ii) muscular tissue,
(iii) skin or feather tissue, and
(iv) organ tissue, said organ tissue preferably being hepatic and / or pancreatic tissue.

The individual test subject, or the individual group of test subjects, are preferably animals, such as invertebrates such as crabs. Alternatively, the individual test subject, or the individual group of test subjects may be vertebrates such as birds or mammals; and preferably are chicken, prawn or crayfish.

The distinct geographic origin may be a geographic location that is considered to be the habitat (including agricultural environments such as a culture farm) wherein the individual test subject, or individual group of test subjects, were spawned and/or cultured, or at least cultured for a significant time during their lifetime.

Preferably, the one or more pre-selected methylation sites are within the 20% most differentially methylated genes of the genome of the individual test subject, or individual group of test subjects.

In a particular example of the first aspect of the present invention, the individual test subject, or the individual group of test subjects is marbled crayfish. Therein, the distinct geographic origins are geographically distinct waters, preferably being selected from the group consisting of lake(s), river(s) and aquaculture farms. These geographically distinct waters may be made distinct from other bodies of water by one or more environmental parameters selected from pH, water hardness, manganese content, iron content, and aluminum content.

The aforementioned method for marbled crayfish advantageously comprises a genome wide methylation analysis or a methylation analysis of a pre-selected panel of methylation sites. These pre-selected panel of methylation sites preferably contain methylation sites within about 500 to 1000, and preferably about 700 genes. The genes or genetic regions according to table 2 are particularly preferred.

In a particular example of the first aspect of the present invention, the individual test subject, or the individual group of test subjects is chicken. Therein, the distinct geographic origins are geographically distinct chicken farms. These geographically distinct chicken farms may be considered distinct from other chicken farms by one or more environmental parameters, such as, feeding parameters or air parameters (e.g. temperature, humidity, ventilation).

Preferably, the panel of methylation sites in the methods according to the first aspect of the present invention does not comprise consistently methylated or unmethylated methylation sites.

In a second aspect, the invention pertains to a method for quality controlling a suspected geographic origin of an individual test subject or individual group of test subjects, the method comprising the steps of
a) determining from the methylation status determined in (a) a test methylation profile of the individual test subject, or of the individual group of test subjects; and
b) comparing the test methylation profile determined in (b) with a predetermined reference methylation profile, wherein the predetermined reference methylation profile is specific for individual subjects, or individual groups of subjects, of the same biological taxon of the individual test subject or individual group of test subjects, and which were obtained from the suspected geographic origin;
wherein if the test methylation profile is significantly similar to the predetermined reference methylation profile, the individual test subject or the individual group of test subjects passes the quality control and the suspected geographical origin is indicated as true geographical origin.

The biological sample containing genomic material may be as defined above.

Also, for this aspect of the present invention, the individual test subject or individual group of test subjects may be any biological entity having a DNA genome and DNA genome methylation. Preferably the methylation site is a CpG site. The individual test subject or individual group of test subjects may be selected from a prokaryote, or a eukaryote, such as a unicellular or multicellular plant, a fungus or an animal. The one or more pre-selected methylation sites in (a) may be methylation sites associated with tissue specific gene expression. Preferably, the pre-selected methylation sites are associated with gene expression of one distinct tissue. Suitable tissues are as defined above for the first aspect of the invention.

The individual test subject, or the individual group of test subjects may be plants and animals, are preferably animals, such as invertebrates such as crabs. Alternatively, the individual test subject, or the individual group of test subjects may be vertebrates such as birds or mammals; and preferably are chicken, prawn or crayfish.

The distinct geographic origin may be a geographic location that is considered to be the habitat (including agricultural environments such as a culture farm) wherein the individual test subject, or individual group of test subjects, were spawned and/or cultured, or at least cultured for a significant time during their lifetime.

Preferably, the one or more pre-selected methylation sites are within the 20% most differentially methylated genes of the genome of the individual test subject, or individual group of test subjects.

In a particular example of the second aspect of the present invention, the individual test subject, or the individual group of test subjects is marbled crayfish. Therein, the distinct geographic origins are geographically distinct waters, preferably being selected from the group consisting of lake(s), river(s) and aquaculture farms. These geographically distinct waters may be considered distinct from other waters by one or more environmental parameters selected from pH, water hardness, manganese content, iron content, and aluminum content.

The aforementioned method for marbled crayfish advantageously comprises a genome wide methylation analysis or a methylation analysis of a pre-selected panel of methylation sites. These pre-selected panel of methylation sites preferably contain methylation sites within about 500 to 1000, and preferably about 700 genes. The genes or genetic regions according to table 2 are particularly preferred

In a particular example of the first aspect of the present invention, the individual test subject, or the individual group of test subjects is chicken. Therein, the distinct geographic origins are geographically distinct chicken farms. These geographically distinct chicken farms may be considered distinct from other chicken farms by one or more environmental parameters, such as, feeding parameters or air parameters (e.g. temperature, humidity, ventilation).

Preferably, the panel of methylation sites in the methods according to the second aspect of the present invention does not comprise consistently methylated or unmethylated methylation sites.

In a third aspect, the invention pertains to a method for assessing one or more environmental parameters of a habitat of an individual test subject, or of an individual group of test subjects, the method comprising the steps of
(a) determining the methylation status of one or more pre-selected methylation sites within the genomic material contained in a biological sample obtained from the individual test subject, or of the individual group of test subjects
(b) determining from the methylation status determined in (a) a test methylation profile of the individual test subject, or of the individual group of test subjects; and
(c) comparing the test methylation profile determined in (b) with one or more predetermined reference methylation profiles, wherein the one or more predetermined reference methylation profiles are each specific for individual subjects, or individual groups of subjects, of the same biological taxon (preferably species) of the individual test subject or the individual group of test subjects, and which were each obtained from distinct geographic origins; and wherein the distinct geographic origin is distinguished from other distinct geographic origins by one or more environmental parameters;
wherein if the test methylation profile is significantly similar to one of the one or more predetermined reference methylation profiles, the individual test subject or individual group of test subjects is derived from a geographical origin having similar, or preferably equal, environmental parameters to the geographical origin of the subjects or group of subjects of the one of the one or more predetermined reference methylation profiles.

The biological sample containing genomic material may be as defined above.

Also, for this aspect of the present invention, the individual test subject or individual group of test subjects may be any biological entity having a DNA genome and DNA genome methylation. Preferably the methylation site is a CpG site. The individual test subject or individual group of test subjects may be selected from a prokaryote, or a eukaryote, such as a unicellular or multicellular plant, a fungus or an animal. The one or more pre-selected methylation sites in (b) may be methylation sites associated with tissue specific gene expression. Preferably, the pre-selected methylation sites are associated with gene expression of one distinct tissue. Suitable tissues are as defined above for the first aspect of the invention.

The individual test subject, or the individual group of test subjects may be plants or animals, are preferably animals, such as invertebrates such as crabs. Alternatively, the individual test subject, or the individual group of test subjects may be vertebrates such as birds or mammals; and preferably are chicken, prawn or crayfish.

The distinct geographic origin may be a geographic location that is considered to be the habitat (including agricultural environments such as a culture farm) wherein the individual test subject, or individual group of test subjects, were spawned and/or cultured, or at least cultured for a significant time during their lifetime.

Preferably, the one or more pre-selected methylation sites are within the 20% most differentially methylated genes of the genome of the individual test subject, or individual group of test subjects.

In a particular example of the third aspect of the present invention, the individual test subject, or the individual group of test subjects is marbled crayfish. Therein, the distinct geographic origins are geographically distinct waters, preferably being selected from the group consisting of lake(s), river(s) and aquaculture farms. These geographically distinct waters may be considered distinct from other bodies of water by one or more environmental parameters selected from pH, water hardness, manganese content, iron content, and aluminum content.

The aforementioned method for marbled crayfish advantageously comprises a genome wide methylation analysis or a methylation analysis of a pre-selected panel of methylation sites. These pre-selected panel of methylation sites preferably contain methylation sites within about 500 to 1000, and preferably about 700 genes. The genes or genetic regions according to table 2 are particularly preferred.

In a particular example of the first aspect of the present invention, the individual test subject, or the individual group of test subjects is chicken. Therein, the distinct geographic origins are geographically distinct chicken farms. These geographically distinct chicken farms may be considered distinct from other chicken farms by one or more environmental parameters, such as, feeding parameters or air parameters (e.g. temperature, humidity, ventilation).

Preferably, the panel of methylation sites in the methods according to the third aspect of the present invention does not comprise consistently methylated or unmethylated methylation sites.

In a fourth aspect, the invention pertains to a method for confirming or declining an assumed geographic origin of an individual test subject or of an individual group of test subjects, the method comprising the comparison of a test methylation profile obtained from genomic material of the individual test subject or of the individual group of test subjects with one or more predetermined reference methylation profiles each being specific for a distinct geographic origin.

The biological sample containing genomic material may be as defined above.

Also, for this aspect of the present invention, the individual test subject or individual group of test subjects may be any biological entity having a DNA genome and DNA genome methylation. Preferably the methylation site is a CpG site. The individual test subject or individual group of test subjects may be selected from a prokaryote, or a eukaryote, such as a unicellular or multicellular plant, a fungus or an animal. The one or more pre-selected methylation sites in (b) may be methylation sites associated with tissue specific gene expression. Preferably, the pre-selected methylation sites are associated with gene expression of one distinct tissue. Suitable tissues are as defined above for the first aspect of the invention.

The individual test subject, or the individual group of test subjects may be plants or animals, are preferably animals, such as invertebrates such as crabs. Alternatively, the individual test subject, or the individual group of test subjects may be vertebrates such as birds or mammals; and preferably are chicken, prawn or crayfish.

The distinct geographic origin may be a geographic location that is considered to be the habitat (including agricultural environments such as a culture farm) wherein the individual test subject, or individual group of test subjects, were spawned and/or cultured, or at least cultured for a significant time during their lifetime.

Preferably, the one or more pre-selected methylation sites are within the 20% most differentially methylated genes of the genome of the individual test subject, or individual group of test subjects.

In a particular example of the fourth aspect of the present invention, the individual test subject, or the individual group of test subjects is marbled crayfish. Therein, the distinct geographic origins are geographically distinct waters, preferably being selected from the group consisting of lake(s), river(s) and aquaculture farms. These geographically distinct waters may be considered distinct from other bodies of water by one or more environmental parameters selected from pH, water hardness, manganese content, iron content, and aluminum content.

The aforementioned method for marbled crayfish advantageously comprises a genome wide methylation analysis or a methylation analysis of a pre-selected panel of methylation sites. These pre-selected panel of methylation sites preferably contain methylation sites within about 500 to 1000, and preferably about 700 genes. The genes or genetic regions according to table 2 are particularly preferred.

In a particular example of the first aspect of the present invention, the individual test subject, or the individual group of test subjects is chicken. Therein, the distinct geographic origins are geographically distinct chicken farms. These geographically distinct chicken farms may be considered distinct from other chicken farms by one or more environmental parameters, such as, feeding parameters or air parameters (e.g. temperature, humidity, ventilation).

Preferably, the panel of methylation sites in the methods according to the fourth aspect of the present invention does not comprise consistently methylated or unmethylated methylation sites.

In a fifth aspect, the invention pertains to a method for developing a test system for confirming an assumed geographic origin of an individual test subject or of an individual group of test subjects, the method comprising the steps of:
a. determining the methylation status of one or more methylation sites within genomic material contained in a biological sample obtained from the individual test subject, or of the individual group of test subjects;
b. selecting from the one or more methylation sites a reference panel of methylation sites which is characterized by a specific and distinct differential methylation profile for each of the known geographic origins;
c. obtaining a test system by assigning a reference methylation profile for each of the known geographic origins (or locations); and
wherein a comparison of a test methylation profile obtained from a test sample with the reference methylation profiles obtained in (c) allows for confirming the assumed geographic origin of the individual test subject or of the individual group of test subjects from which the test sample was obtained.

The biological sample containing genomic material may be as defined above.

Also, for this aspect of the present invention, the individual test subject or individual group of test subjects may be any biological entity having a DNA genome and DNA genome methylation. Preferably the methylation site is a CpG site. The individual test subject or individual group of test subjects may be selected from a prokaryote, or a eukaryote, such as a unicellular or multicellular plant, a fungus or an animal. The one or more pre-selected methylation sites may be methylation sites associated with tissue specific gene expression. Preferably, the pre-selected methylation sites are associated with gene expression of one distinct tissue. Suitable tissues are as defined above for the first aspect of the invention.

The individual test subject, or the individual group of test subjects, are preferably animals, such as invertebrates such as crabs. Alternatively, the individual test subject, or the individual group of test subjects may be vertebrates such as birds or mammals; and preferably are chicken, prawn or crayfish.

The distinct geographic origin may be a geographic location that is considered to be the habitat (including agricultural environments such as a culture farm) wherein the individual test subject, or individual group of test subjects, were spawned and/or cultured, or at least cultured for a significant time during their lifetime.

Preferably, the one or more pre-selected methylation sites are within the 20% most differentially methylated genes of the genome of the individual test subject, or individual group of test subjects.

In a particular example of the second aspect of the present invention, the individual test subject, or the individual group of test subjects is marbled crayfish. Therein, the distinct geographic origins are geographically distinct waters, preferably being selected from the group consisting of lake(s), river(s) and aquaculture farms. These geographically distinct waters may be considered distinct from other bodies of water by one or more environmental parameters selected from pH, water hardness, manganese content, iron content, and aluminum content.

The aforementioned method for marbled crayfish advantageously comprises a genome wide methylation analysis or a methylation analysis of a pre-selected panel of methylation sites. These pre-selected panel of methylation sites preferably contain methylation sites within about 500 to 1000, and preferably about 700 genes. The genes or genetic regions according to table 2 are particularly preferred.

In a particular example of the first aspect of the present invention, the individual test subject, or the individual group of test subjects is chicken. Therein, the distinct geographic origins are geographically distinct chicken farms. These geographically distinct chicken farms may be considered to be distinct from other chicken farms by one or more environmental parameters, such as, feeding parameters or air parameters (e.g. temperature, humidity, ventilation).

Preferably, the panel of methylation sites in the methods according to the fifth aspect of the present invention does not comprise consistently methylated or unmethylated methylation sites.

### BRIEF DESCRIPTION OF THE FIGURES

FIG. 1 shows specific water parameters of four Marbled crayfish population habitats.

FIG. 2 shows context-specific differential methylation in marbled crayfish populations. (A) Principal component analysis of abdominal muscle (mus., square symbols) and hepatopancreas (hep., circular symbols) samples from Singlis, based on the methylation levels of 56 genes with tissue-specific methylation differences. (B) Principal component analysis of abdominal muscle (mus., square symbols) and hepatopancreas (hep., circular symbols) samples from Reilingen, based on the methylation levels of 35 genes with tissue-specific methylation differences. (C) Principal component analysis of hepatopancreas samples from all locations, based on the methylation levels of 122 genes with location-specific methylation differences. (D) Principal component analysis of abdominal muscle samples from all locations, based on the methylation levels of 22 genes with location-specific methylation differences.

FIG. 3 shows the validation of context-dependent differential methylation in marbled crayfish. Results are shown for capture-based sequencing and for the corresponding validation experiment with amplicon sequencing, for 4 different genomic regions. Unfilled shapes: abdominal muscle; filled shapess: hepatopancreas;squares: Reilingen; stars: Singlis; circles: Andragnaroa; triangle: Ihosy.

FIG. 4 are the results of differentially methylated CpG sites in chicken using the function “calculate DiffMeth” from the R package MethylKit on Reduced representation bisulfite sequencing (RRBS) data. The identified differentially methylated CpG sites allowed a robust separation of the three locations in a principle component analysis. After filtering for SNPs: 2.3 - 3.6 million CpG sites. CpG sites with min coverage 10 in all the samples: 623,657, Differentially methylated CpGs:1274 (p-value <0.05).

FIG. 5 are the results of differentially methylated CpG sites in soho salmon using the function “calculate DiffMeth” from the R package MethylKit on Reduced representation bisulfite sequencing (RRBS) data. The identified differentially methylated CpG sites allowed a robust separation of the two locations in a principle component analysis. CpG sites with min coverage 10 in all the samples after SNP filtering: 610,397, Significant DMRs: 440 (p-value <0.05, diff in methylation>=10%)

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

### Example 1

### Habitat Profiles of Four Independent Marbled Crayfish Populations

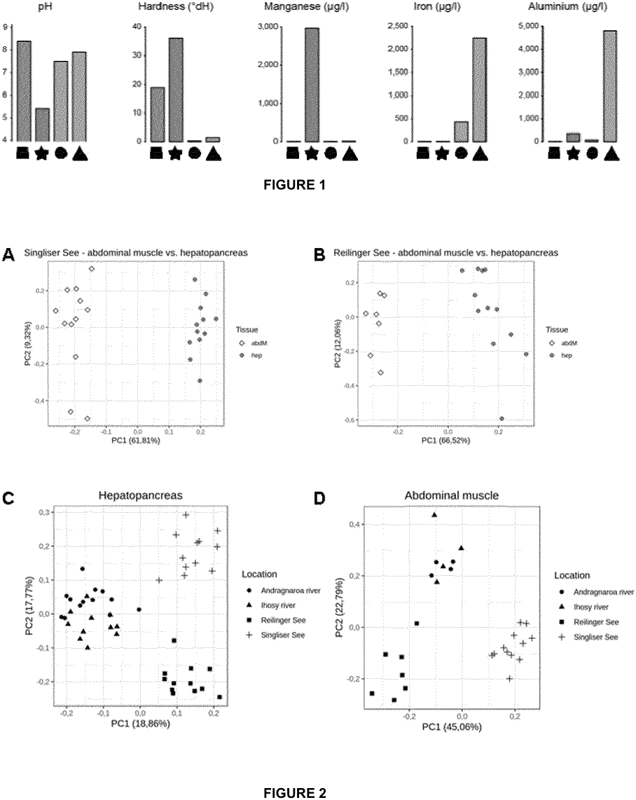

To explore the possibility of context-dependent DNA methylation in marbled crayfish, animals from four diverse stable populations were collected. Reilingen (Germany) represents the type locality, a small eutrophic lake in an environmentally protected area. The Singlis (Germany) population is from a larger oligotrophic lake with in a former brown coal mining area. The Andragnaroa (Madagascar) population is located in a river flowing through a forest area at relatively high altitude (1156 m) with soft mountain water. Finally, the Ihosy (Madagascar) population is found in highly turbid water, with high levels of pollution from nearby mining activities. The analysis of physicochemical water parameters showed clean, slightly basic (pH 8.4) water in Reilingen and rather acidic (pH 5.2) water with high levels of Manganese (4792 µg/l) in Singlis. The water in Andragnaroa showed particularly low hardness (0.3 °dH), while the water in Ihosy was characterized by high levels of Aluminium (2967 µg/l) and Iron (2249 µg/l). Altogether, our study thus covered populations that inhabit four diverse habitats from different climatic zones and with different water parameters. These results are shown in FIG. 1,

TABLE 1

Overview of marbled crayfish populations analyzed

| Geographic location (site name) | Coordinates | Type | Altitude (m) | Key features | Ground sediment | Associated vegetation and fauna |
|---|---|---|---|---|---|---|
| Reilingen (Germany) | N49°17,649′ E08°32,672′ | lake | 69 | eutrophic lake | mud, sand | herbaceous grasses, macrophytes, algae, fish, insects, crayfish |
| Singlis (Germany) | N51°03.655′ E09°18.710′ | lake | 168 | oligotrophic lake, acidic water | sand, pebbles | herbaceous grasses, insects |
| Andragnaroa (Madagascar) | S21°17.551′ E47°22.292′ | river | 1083 | slow-flowing mountain river | mud | herbaceous grasses, rice, fish, insects, crabs, crayfish |
| Ihosy (Madagascar) | S22°22.512′ E46°06.016′ | river | 711 | slow-flowing, turbid, polluted river | mud | herbaceous grasses, fish, amphibians, molluscs, insects |

### Example 2

### Identification of a Variably Methylated Gene Set

It was previously shown that DNA methylation in the marbled crayfish is targeted to gene bodies, relatively stable and largely tissue-invariant (Gatzmann et al., 2018). However, a comparison of 8 whole-genome bisulfite sequencing datasets from different animals, different tissues and different developmental stages also indicated the possibility for a smaller group of genes that showed more variable methylation levels (Gatzmann et al., 2018). This was confirmed by systematic analyses of methylation variance. A variance cutoff of >0.006 identified 846 genes, 149 of which were consistently methylated or unmethylated (mean ratio >0.8 or <0.2, respectively) and excluded from further analysis, thus defining a core set of 697 variably methylated genes. Metric multidimensional analysis based on the methylation levels of these genes separated the hepatopancreas samples from the abdominal muscle samples, which suggested the presence of previously unrecognized tissue-specific methylation patterns.

In order to analyze the methylation patterns of these genes in a larger number of samples and at higher coverage methylation, a bead-based capture assay was developed. For this assay, DNA samples from 2 different tissues were prepared: hepatopancreas, which represents the main metabolic organ of crayfish and abdominal muscle, the main muscle tissue forming the abdominal tail. Hepatopancreas DNA was prepared from N=47 animals (11-12 per location), while abdominal muscle DNA was prepared from a subset of the same animals (N=26, 12-4 per location). Subgenome capture was found to be both efficient and specific, providing a minimum of 10 million mapped reads per sample under stringent conditions.

In subsequent steps, genes with more than 50% Ns in their sequence were excluded, which left 623 genes in our analysis. Furthermore, only those CpG sites that were present in all the samples with a sequencing coverage of ≥5x were considered and average methylation levels were calculated only if a gene had ≥5 qualified CpG sites. These criteria were fulfilled for 463 genes. The inventors also excluded invariant genes, i.e., genes that were in the bottom 10% for methylation variance as well as genes with an average methylation level <0.1 or >0.9, resulting in a core set of 361 variably methylated genes (Tab. 2).

TABLE 2

Genomic regions suitable as methylation markers in marbled crayfish

| gene_id | chr | start | end |
|---|---|---|---|
| maker-scaffold304068-snap-gene-0.0 | scaffold304068 | 1337 | 27574 |
| snap_masked-scaffold24197-processed-gene-0.0 | scaffold24197 | 8904 | 43369 |
| snap-scaffold36687-processed-gene-0.8 | scaffold36687 | 137868 | 162515 |
| snap_masked-scaffold90387-processed-gene-0.16 | scaffold90387 | 50002 | 65769 |
| evm-scaffold108432-processed-gene-0.3 | scaffold108432 | 65051 | 76801 |
| evm-scaffold139595-processed-gene-0.11 | scaffold139595 | 4000 | 19145 |
| snap-scaffold26860-processed-gene-0.5 | scaffold26860 | 113376 | 137381 |
| evm-scaffold16904-processed-gene-1.0 | scaffold16904 | 183886 | 196760 |
| maker-scaffold10264-snap-gene-0.18 | scaffold10264 | 25066 | 37578 |
| maker-scaffold9659-snap-gene-1.19 | scaffold9659 | 203904 | 211046 |
| maker-scaffold2381-snap-gene-1.5 | scaffold2381 | 83970 | 96356 |
| evm-scaffold50337-processed-gene-0.4 | scaffold50337 | 54275 | 66946 |
| maker-scaffold45362-snap-gene-0.0 | scaffold45362 | 65031 | 78444 |
| maker-scaffold115264-snap-gene-0.3 | scaffold115264 | 19872 | 31054 |
| maker-scaffold10188-snap-gene-0.1 | scaffold10188 | 54147 | 60918 |
| snap_masked-scaffold50797-processed-gene-0.7 | scaffold50797 | 37447 | 42476 |
| snap-scaffold115264-processed-gene-0.9 | scaffold115264 | 38152 | 63093 |
| maker-scaffold11552-snap-gene-2.41 | scaffold11552 | 256598 | 273594 |
| maker-scaffold126600-snap-gene-0.20 | scaffold126600 | 85747 | 92192 |
| evm-scaffold12945-processed-gene-0.21 | scaffold12945 | 14168 | 20265 |
| snap_masked-scaffold93376-processed-gene-0.9 | scaffold93376 | 16276 | 32089 |
| maker-scaffold219941-snap-gene-0.1 | scaffold219941 | 2898 | 11055 |
| maker-scaffold15530-snap-gene-0.12 | scaffold15530 | 70666 | 87866 |
| maker-scaffold12744-snap-gene-1.27 | scaffold12744 | 114212 | 127348 |
| maker-scaffold8191-snap-gene-0.0 | scaffold8191 | 48342 | 67985 |
| maker-scaffold175420-snap-gene-0.0 | scaffold175420 | 16768 | 32937 |
| evm-scaffold112413-processed-gene-0.17 | scaffold112413 | 25163 | 31291 |
| snap-scaffold39846-processed-gene-0.9 | scaffold39846 | 18870 | 30259 |
| maker-scaffold121213-snap-gene-0.1 | scaffold121213 | 30065 | 35437 |
| snap_masked-scaffold43456-processed-gene-0.8 | scaffold43456 | 30046 | 39826 |
| maker-scaffold17132-snap-gene-0.32 | scaffold17132 | 3351 | 27102 |
| maker-scaffold267215-snap-gene-0.0 | scaffold267215 | 7481 | 13107 |
| maker-scaffold205616-snap-gene-0.0 | scaffold205616 | 49312 | 53787 |
| snap-scaffold53412-processed-gene-0.5 | scaffold53412 | 59522 | 68472 |
| maker-scaffold135435-snap-gene-0.1 | scaffold135435 | 249 | 9302 |
| snap-scaffold4868-processed-gene-0.30 | scaffold4868 | 36318 | 50961 |
| evm-scaffold41057-processed-gene-0.1 | scaffold41057 | 28601 | 33526 |
| maker-scaffold102285-snap-gene-0.10 | scaffold102285 | 38482 | 46524 |
| maker-scaffold220173-snap-gene-0.0 | scaffold220173 | 1241 | 9258 |
| maker-scaffold91737-snap-gene-0.0 | scaffold91737 | 39280 | 44975 |
| maker-scaffold6474-snap-gene-0.6 | scaffold6474 | 33723 | 47661 |
| evm-scaffold33165-processed-gene-0.3 | scaffold33165 | 58807 | 65868 |
| snap-scaffold8703-processed-gene-0.1 | scaffold8703 | 39503 | 43579 |
| maker-scaffold48239-snap-gene-0.18 | scaffold48239 | 64621 | 72046 |
| maker-scaffold32877-snap-gene-0.1 | scaffold32877 | 8946 | 23196 |
| maker-scaffold1498-snap-gene-0.3 | scaffold1498 | 57051 | 67352 |
| evm-scaffold94418-processed-gene-0.14 | scaffold94418 | 53835 | 60225 |
| maker-scaffold13345-snap-gene-1.11 | scaffold13345 | 82911 | 91955 |
| snap_masked-scaffold74137-processed-gene-0.3 | scaffold74137 | 17995 | 21318 |
| maker-scaffold50170-snap-gene-0.19 | scaffold50170 | 34890 | 40929 |
| evm-scaffold43820-processed-gene-0.1 | scaffold43820 | 71976 | 78177 |
| evm-scaffold172683-processed-gene-0.3 | scaffold172683 | 67195 | 72070 |
| maker-scaffold263285-snap-gene-0.1 | scaffold263285 | 22636 | 31057 |
| maker-scaffold123276-snap-gene-0.16 | scaffold123276 | 48317 | 60296 |
| maker-scaffold113704-exonerate_est2genome-gene-0.17 | scaffold113704 | 682 | 1469 |
| maker-scaffold4620-snap-gene-0.26 | scaffold4620 | 11979 | 20871 |
| maker-scaffold7189-snap-gene-0.3 | scaffold7189 | 19816 | 28919 |
| evm-scaffold16727-processed-gene-0.11 | scaffold16727 | 63585 | 71191 |
| maker-scaffold12256-snap-gene-0.0 | scaffold12256 | 28180 | 36440 |
| evm-scaffold397263-processed-gene-0.0 | scaffold397263 | 26651 | 30566 |
| evm-scaffold9304-processed-gene-0.27 | scaffold9304 | 97512 | 103845 |
| maker-scaffold114487-snap-gene-0.3 | scaffold114487 | 141172 | 149611 |
| maker-scaffold48239-exonerate_est2genome-gene-0.1 | scaffold48239 | 72267 | 72884 |
| maker-scaffold10961-snap-gene-0.5 | scaffold10961 | 464 | 7461 |
| evm-scaffold100674-processed-gene-0.5 | scaffold100674 | 62519 | 66202 |
| evm-scaffold9911-processed-gene-0.23 | scaffold9911 | 57148 | 61973 |
| maker-scaffold101782-snap-gene-0.0 | scaffold101782 | 359 | 3823 |
| evm-scaffold5511-processed-gene-0.0 | scaffold5511 | 19862 | 25147 |
| snap_masked-scaffold310636-processed-gene-0.1 | scaffold310636 | 12641 | 14932 |
| maker-scaffold13666-snap-gene-0.25 | scaffold13666 | 93821 | 101729 |
| maker-scaffold38912-snap-gene-0.1 | scaffold38912 | 35958 | 42540 |
| maker-scaffold38310-snap-gene-0.19 | scaffold38310 | 26015 | 28730 |
| evm-scaffold6249-processed-gene-0.16 | scaffold6249 | 13015 | 18415 |
| maker-scaffold124456-snap-gene-0.10 | scaffold124456 | 40484 | 46419 |
| maker-scaffold12620-snap-gene-0.21 | scaffold12620 | 879 | 5599 |
| maker-scaffold48310-snap-gene-0.0 | scaffold48310 | 8226 | 11931 |
| evm-scaffold34440-processed-gene-0.36 | scaffold34440 | 83604 | 88687 |
| maker-scaffold71508-snap-gene-0.7 | scaffold71508 | 1687 | 7045 |
| snap-scaffold6152-processed-gene-0.21 | scaffold6152 | 110089 | 114729 |
| maker-scaffold52598-snap-gene-0.3 | scaffold52598 | 4758 | 12239 |
| maker-scaffold54060-exonerate_est2genome-gene-0.2 | scaffold54060 | 7844 | 12054 |
| evm-scaffold39916-processed-gene-0.41 | scaffold39916 | 152669 | 158190 |
| maker-scaffold9999-snap-gene-0.39 | scaffold9999 | 123755 | 131121 |
| snap-scaffold14680-processed-gene-0.21 | scaffold14680 | 76788 | 82577 |
| maker-scaffold28267-snap-gene-0.0 | scaffold28267 | 7743 | 13738 |
| maker-scaffold394459-snap-gene-0.5 | scaffold394459 | 1518 | 8604 |
| evm-scaffold90817-processed-gene-0.1 | scaffold90817 | 9485 | 13683 |
| evm-scaffold371305-processed-gene-0.0 | scaffold371305 | 17158 | 21261 |
| maker-scaffold130709-exonerat_est2genome-gene-0.10 | scaffold130709 | 6192 | 13241 |
| maker-scaffold11851-snap-gene-0.5 | scaffold11851 | 77 | 5252 |
| maker-scaffold22339-snap-gene-0.0 | scaffold22339 | 1122 | 5657 |
| evm-scaffold107110-processed-gene-0.0 | scaffold107110 | 986 | 2634 |
| evm-scaffold73810-processed-gene-1.35 | scaffold73810 | 67198 | 69697 |
| evm-scaffold40617-processed-gene-0.7 | scaffold40617 | 42743 | 47819 |
| evm-scaffold137559-processed-gene-0.22 | scaffold137559 | 63163 | 67788 |
| maker-scaffold202891-snap-gene-0.5 | scaffold202891 | 428 | 4466 |
| snap_masked-scaffold81770-processed-gene-0.17 | scaffold81770 | 87096 | 89144 |
| maker-scaffold27888-snap-gene-0.2 | scaffold27888 | 56636 | 64796 |
| maker-scaffold339-snap-gene-1.14 | scaffold339 | 182807 | 188079 |
| evm-scaffold7906-processed-gene-1.0 | scaffold7906 | 90914 | 96317 |
| maker-scaffold564-snap-gene-1.5 | scaffold564 | 110968 | 116601 |
| snap_masked-scaffold104332-processed-gene-0.1 | scaffold104332 | 7495 | 13716 |
| maker-scaffold5412-snap-gene-1.1 | scaffold5412 | 147667 | 150797 |
| maker-scaffold22213-snap-gene-0.22 | scaffold22213 | 60151 | 68877 |
| maker-scaffold26595-snap-gene-0.19 | scaffold26595 | 32853 | 44683 |
| maker-scaffold23087-snap-gene-0.10 | scaffold23087 | 20936 | 26723 |
| evm-scaffold80512-processed-gene-0.10 | scaffold80512 | 66725 | 75346 |
| maker-scaffold17930-snap-gene-0.0 | scaffold17930 | 74641 | 76992 |
| snap_masked-scaffold868-processed-gene-1.34 | scaffold868 | 141766 | 146382 |
| maker-scaffold6973-snap-gene-0.2 | scaffold6973 | 4987 | 7505 |
| maker-scaffold1857-snap-gene-1.34 | scaffold1857 | 83854 | 91724 |
| snap_masked-scaffold91879-processed-gene-0.2 | scaffold91879 | 17111 | 28264 |
| maker-scaffold386719-snap-gene-0.2 | scaffold386719 | 6768 | 11610 |
| snap-scaffold30198-processed-gene-0.4 | scaffold30198 | 998 | 6259 |
| maker-scaffold16863-snap-gene-0.12 | scaffold16863 | 10901 | 15377 |
| maker-scaffold80517-snap-gene-0.0 | scaffold80517 | 24051 | 29834 |
| evm-scaffold228228-processed-gene-0.1 | scaffold228228 | 48536 | 52576 |
| snap-scaffold102750-processed-gene-0.6 | scaffold102750 | 75430 | 82953 |
| evm-scaffold1978-processed-gene-0.5 | scaffold1978 | 22655 | 29497 |
| evm-scaffold36395-processed-gene-0.8 | scaffold36395 | 9144 | 14617 |
| evm-scaffold59094-processed-gene-0.23 | scaffold59094 | 68984 | 73308 |
| evm-scaffold48548-processed-gene-0.0 | scaffold48548 | 17748 | 20389 |
| maker-scaffold377919-snap-gene-0.0 | scaffold377919 | 34891 | 42885 |
| snap-scaffold74799-processed-gene-0.5 | scaffold74799 | 75543 | 76292 |
| evm-scaffold74849-processed-gene-1.29 | scaffold74849 | 177285 | 182531 |
| snap_masked-scaffold59159-processed-gene-0.9 | scaffold59159 | 49876 | 50094 |
| snap_masked-scaffold2177-processed-gene-0.6 | scaffold2177 | 129902 | 135993 |
| evm-scaffold361614-processed-gene-0.1 | scaffold361614 | 8789 | 14371 |
| maker-scaffold81285-snap-gene-0.0 | scaffold81285 | 23168 | 25422 |
| maker-scaffold107280-snap-gene-0.0 | scaffold107280 | 19587 | 22364 |
| snap-scaffold111395-processed-gene-0.7 | scaffold111395 | 39120 | 45694 |
| maker-scaffold4989-snap-gene-0.21 | scaffold4989 | 47361 | 52650 |
| snap-scaffold61385-processed-gene-0.6 | scaffold61385 | 38072 | 39592 |
| evm-scaffold35783-processed-gene-0.1 | scaffold35783 | 25675 | 32243 |
| maker-scaffold50170-exonerate_est2genome-gene-0.0 | scaffold50170 | 33956 | 34825 |
| maker-scaffold38451-snap-gene-0.0 | scaffold38451 | 38756 | 45073 |
| snap_masked-scaffold25208-processed-gene-0.0 | scaffold25208 | 12 | 486 |
| maker-scaffold138460-exonerate_est2genome-gene-0.45 | scaffold138460 | 111216 | 111777 |
| snap-scaffold53368-processed-gene-0.1 | scaffold53368 | 11351 | 12349 |
| snap-scaffold16922-processed-gene-0.14 | scaffold16922 | 144576 | 147649 |
| maker-scaffold3650-snap-gene-0.0 | scaffold3650 | 51947 | 56482 |
| maker-scaffold112453-snap-gene-0.2 | scaffold112453 | 94164 | 97264 |
| maker-scaffold41290-snap-gene-2.1 | scaffold41290 | 227621 | 232155 |
| maker-scaffold10925-exonerate_est2genome-gene-0.28 | scaffold10925 | 43088 | 44269 |
| maker-scaffold3354-snap-gene-0.1 | scaffold3354 | 14246 | 19146 |
| snap-scaffold45749-processed-gene-0.6 | scaffold45749 | 28428 | 31630 |
| snap-scaffold81425-processed-gene-0.9 | scaffold81425 | 26428 | 35106 |
| maker-scaffold23229-snap-gene-1.15 | scaffold23229 | 109617 | 113443 |
| maker-scaffold73264-snap-gene-0.0 | scaffold73264 | 6157 | 8104 |
| snap_masked-scaffold62530-processed-gene-0.4 | scaffold62530 | 16714 | 18750 |
| snap-scaffold5751-processed-gene-0.4 | scaffold5751 | 29224 | 29448 |
| maker-scaffold59094-snap-gene-0.22 | scaffold59094 | 85362 | 87038 |
| maker-scaffold211263-snap-gene-0.11 | scaffold211263 | 40503 | 43319 |
| maker-scaffold25493-snap-gene-0.48 | scaffold25493 | 33080 | 37341 |
| maker-scaffold76097-snap-gene-0.13 | scaffold76097 | 61195 | 63396 |
| maker-scaffold1180-snap-gene-0.9 | scaffold1180 | 72593 | 78002 |
| maker-scaffold31717-snap-gene-0.2 | scaffold31717 | 60581 | 68418 |
| maker-scaffold44746-snap-gene-0.0 | scaffold44746 | 66445 | 71453 |
| evm-scaffold22394-processed-gene-2.5 | scaffold22394 | 251018 | 254621 |
| snap_masked-scaffold9798-processed-gene-0.0 | scaffold9798 | 21268 | 21624 |
| maker-scaffold215670-snap-gene-0.0 | scaffold215670 | 5627 | 11303 |
| maker-scaffold21855-snap-gene-0.4 | scaffold21855 | 132449 | 136040 |
| maker-scaffold61175-snap-gene-0.20 | scaffold61175 | 47087 | 48344 |
| snap_masked-scaffold5220-processed-gene-1.12 | scaffold5220 | 154619 | 155515 |
| maker-scaffold72239-snap-gene-0.8 | scaffold72239 | 4943 | 8293 |
| snap-scaffold27036-processed-gene-0.0 | scaffold27036 | 18815 | 19618 |
| snap-scaffold122449-processed-gene-0.0 | scaffold122449 | 1099 | 1506 |
| maker-scaffold41290-snap-gene-1.0 | scaffold41290 | 94934 | 98362 |
| maker-scaffold156213-snap-gene-1.20 | scaffold156213 | 106417 | 108341 |
| maker-scaffold39916-snap-gene-0.48 | scaffold39916 | 147719 | 152559 |
| snap-scaffold1620-processed-gene-1.39 | scaffold1620 | 229567 | 233057 |
| maker-scaffold10917-snap-gene-0.1 | scaffold10917 | 99892 | 101179 |
| evm-scaffold39916-processed-gene-0.39 | scaffold39916 | 115273 | 119446 |
| maker-scaffold8594-snap-gene-0.3 | scaffold8594 | 161003 | 165873 |
| maker-scaffold156352-snap-gene-0.0 | scaffold156352 | 4759 | 8791 |
| maker-scaffold262363-snap-gene-0.0 | scaffold262363 | 25460 | 29529 |
| snap_masked-scaffold41199-processed-gene-0.3 | scaffold41199 | 28695 | 29186 |
| maker-scaffold2625-exonerate_est2genome-gene-1.48 | scaffold2625 | 169586 | 173199 |
| snap-scaffold135378-processed-gene-0.13 | scaffold135378 | 80922 | 85145 |
| evm-scaffold9975-processed-gene-1.28 | scaffold9975 | 92463 | 98507 |
| snap-scaffold135539-processed-gene-0.4 | scaffold135539 | 36766 | 37365 |
| snap-scaffold70321-processed-gene-0.9 | scaffold70321 | 72790 | 73173 |
| evm-scaffold56737-processed-gene-0.25 | scaffold56737 | 33595 | 36872 |
| evm-scaffold49405-processed-gene-0.2 | scaffold49405 | 57239 | 60293 |
| snap_masked-scaffold19330-processed-gene-0.11 | scaffold19330 | 46109 | 46777 |
| snap_masked-scaffold23847-processed-gene-0.23 | scaffold23847 | 106662 | 107048 |
| snap-scaffold5583-processed-gene-1.21 | scaffold5583 | 141290 | 141757 |
| snap-scaffold5020-processed-gene-0.4 | scaffold5020 | 37952 | 38401 |
| snap-scaffold116111-processed-gene-0.3 | scaffold116111 | 14899 | 15399 |
| snap-scaffold7627-processed-gene-0.4 | scaffold7627 | 45053 | 45893 |
| snap-scaffold91170-processed-gene-0.1 | scaffold91170 | 764 | 1429 |
| maker-scaffold12911-snap-gene-0.5 | scaffold12911 | 69371 | 71899 |
| snap-scaffold352968-processed-gene-0.0 | scaffold352968 | 568 | 1035 |
| snap-scaffold19330-processed-gene-0.4 | scaffold19330 | 26274 | 28769 |
| snap-scaffold52698-processed-gene-0.12 | scaffold52698 | 39460 | 39846 |
| maker-scaffold16344-exonerate_est2genome-gene-0.22 | scaffold16344 | 54299 | 56148 |
| maker-scaffold18679-snap-gene-0.48 | scaffold18679 | 92344 | 92876 |
| snap-scaffold257007-processed-gene-0.6 | scaffold257007 | 27732 | 28088 |
| snap_masked-scaffold522-processed-gene-0.3 | scaffold522 | 50041 | 50616 |
| snap-scaffold5124-processed-gene-0.4 | scaffold5124 | 12695 | 12982 |
| maker-scaffold25095-snap-gene-0.69 | scaffold25095 | 63863 | 64998 |
| snap-scaffold32024-processed-gene-0.3 | scaffold32024 | 24648 | 24866 |
| evm-scaffold83705-processed-gene-0.1 | scaffold83705 | 25046 | 28714 |
| evm-scaffold134054-processed-gene-0.11 | scaffold134054 | 29553 | 32804 |
| evm-scaffold57-processed-gene-1.48 | scaffold57 | 104482 | 108289 |
| snap-scaffold52598-processed-gene-0.25 | scaffold52598 | 107050 | 107586 |
| snap-scaffold21794-processed-gene-0.26 | scaffold21794 | 69850 | 70434 |
| snap_masked-scaffold22145-processed-gene-0.1 | scaffold22145 | 688 | 954 |
| snap_masked-scaffold87134-processed-gene-0.3 | scaffold87134 | 23056 | 23358 |
| snap-scaffold54195-processed-gene-0.39 | scaffold54195 | 98175 | 98477 |
| snap_masked-scaffold18008-processed-gene-0.1 | scaffold18008 | 19654 | 20070 |
| maker-scaffold333883-exonerate_est2genome-gene-0.0 | scaffold333883 | 9208 | 9684 |
| snap_masked-scaffold140642-processed-gene-0.7 | scaffold140642 | 10935 | 11473 |
| maker-scaffold140642-exonerate_est2genome-gene-0.0 | scaffold140642 | 11139 | 11740 |
| evm-scaffold10046-processed-gene-0.0 | scaffold10046 | 61937 | 64677 |
| maker-scaffold11617-snap-gene-0.34 | scaffold11617 | 27592 | 31834 |
| snap-scaffold140713-processed-gene-0.3 | scaffold140713 | 31608 | 38022 |
| snap_masked-scaffold98835-processed-gene-0.5 | scaffold98835 | 34867 | 35255 |
| snap-scaffold35469-processed-gene-0.3 | scaffold35469 | 36010 | 36411 |
| maker-scaffold117568-exonerate_est2genome-gene-0.7 | scaffold117568 | 15868 | 16247 |
| evm-scaffold742-processed-gene-0.36 | scaffold742 | 61057 | 63185 |
| evm-scaffold4470-processed-gene-1.4 | scaffold4470 | 120489 | 122455 |
| maker-scaffold46239-snap-gene-0.1 | scaffold46239 | 87878 | 90794 |
| snap-scaffold3259-processed-gene-1.3 | scaffold3259 | 50485 | 50827 |
| snap-scaffold317362-processed-gene-0.1 | scaffold317362 | 1192 | 1482 |
| snap-scaffold10188-processed-gene-0.18 | scaffold10188 | 27890 | 29985 |
| snap-scaffold122226-processed-gene-0.3 | scaffold122226 | 40393 | 40945 |
| snap-scaffold50170-processed-gene-0.7 | scaffold50170 | 1950 | 2341 |
| snap_masked-scaffold207763-processed-gene-0.2 | scaffold207763 | 17887 | 18698 |
| snap_masked-scaffold92118-processed-gene-0.3 | scaffold92118 | 11370 | 11660 |
| snap-scaffold168208-processed-gene-0.0 | scaffold168208 | 855 | 1424 |
| maker-scaffold134109-snap-gene-0.14 | scaffold134109 | 39275 | 41980 |
| maker-scaffold6421-snap-gene-0.31 | scaffold6421 | 36942 | 39630 |
| maker-scaffold60601-exonerate_est2genome-gene-0.20 | scaffold60601 | 11934 | 12862 |
| maker-scaffold97830-snap-gene-0.2 | scaffold97830 | 18417 | 18937 |
| snap-scaffold5315-processed-gene-0.29 | scaffold5315 | 45483 | 45707 |
| snap-scaffold28753-processed-gene-0.18 | scaffold28753 | 78018 | 78470 |
| snap_masked-scaffold367392-processed-gene-0.11 | scaffold367392 | 7787 | 8014 |
| snap-scaffold49466-processed-gene-0.4 | scaffold49466 | 2519 | 2848 |
| snap-scaffold392560-processed-gene-0.4 | scaffold392560 | 11902 | 12204 |
| snap-scaffold15934-processed-gene-0.3 | scaffold15934 | 149781 | 150110 |
| snap_masked-scaffold18992-processed-gene-0.6 | scaffold18992 | 46014 | 46271 |
| snap_masked-scaffold146957-processed-gene-0.3 | scaffold146957 | 26384 | 27918 |
| snap-scaffold25878-processed-gene-0.9 | scaffold25878 | 15107 | 15409 |
| snap_masked-scaffold73424-processed-gene-0.1 | scaffold73424 | 7297 | 7599 |
| snap_masked-scaffold97644-processed-gene-0.15 | scaffold97644 | 10259 | 10567 |
| snap_masked-scaffold53654-processed-gene-0.3 | scaffold53654 | 7191 | 7771 |
| maker-scaffold47681-exonerate_est2genome-gene-0.0 | scaffold47681 | 356 | 970 |
| maker-scaffold31708-snap-gene-0.2 | scaffold31708 | 69163 | 73176 |
| maker-scaffold6368-snap-gene-0.42 | scaffold6368 | 101857 | 106342 |
| snap-scaffold75609-processed-gene-0.2 | scaffold75609 | 6101 | 11966 |
| snap_masked-scaffold225859-processed-gene-0.4 | scaffold225859 | 45899 | 46424 |
| snap-scaffold25619-processed-gene-0.14 | scaffold25619 | 11173 | 11799 |
| evm-scaffold13441-processed-gene-0.0 | scaffold13441 | 117539 | 120929 |
| snap_masked-scaffold22208-processed-gene-1.23 | scaffold22208 | 130498 | 130764 |
| snap-scaffold90609-processed-gene-0.36 | scaffold90609 | 47019 | 47240 |
| snap-scaffold157241-processed-gene-0.8 | scaffold157241 | 35342 | 35566 |
| snap_masked-scaffold54060-processed-gene-0.3 | scaffold54060 | 2684 | 3304 |
| snap_masked-scaffold195460-processed-gene-0.3 | scaffold195460 | 39668 | 40474 |
| snap_masked-scaffold10502-processed-gene-0.7 | scaffold10502 | 12267 | 12569 |
| snap_masked-scaffold142074-processed-gene-0.0 | scaffold142074 | 20258 | 20557 |
| snap_masked-scaffold43914-processed-gene-0.1 | scaffold43914 | 42702 | 43364 |
| maker-scaffold16651-exonerate_est2genome-gene-0.0 | scaffold16651 | 73734 | 74441 |
| maker-scaffold44294-exonerate_est2genome-gene-0.1 | scaffold44294 | 896 | 1512 |
| snap-scaffold37344-processed-gene-0.10 | scaffold37344 | 77552 | 78040 |
| snap-scaffold23679-processed-gene-1.15 | scaffold23679 | 210879 | 211460 |
| snap-scaffold5808-processed-gene-1.32 | scaffold5808 | 182568 | 182987 |
| evm-scaffold22787-processed-gene-0.15 | scaffold22787 | 53527 | 53951 |
| snap-scaffold17307-processed-gene-0.2 | scaffold17307 | 2378 | 2863 |
| maker-scaffold7189-exonerate_est2genome-gene-0.9 | scaffold7189 | 88683 | 89274 |
| maker-scaffold43849-exonerate_est2genome-gene-0.19 | scaffold43849 | 61106 | 63365 |
| snap_masked-scaffold61451-processed-gene-0.2 | scaffold61451 | 8144 | 8368 |
| snap-scaffold26326-processed-gene-0.0 | scaffold26326 | 965 | 1421 |
| snap-scaffold182519-processed-gene-0.1 | scaffold182519 | 6486 | 6770 |
| snap_masked-scaffold9248-processed-gene-0.0 | scaffold9248 | 7599 | 8186 |
| maker-scaffold42144-snap-gene-0.3 | scaffold42144 | 68485 | 69224 |
| maker-scaffold30907-exonerate_est2genome-gene-0.43 | scaffold30907 | 78759 | 79432 |
| snap_masked-scaffold12875-processed-gene-0.20 | scaffold12875 | 106918 | 107486 |
| snap_masked-scaffold318945-processed-gene-0.0 | scaffold318945 | 16777 | 17068 |
| snap-scaffold114005-processed-gene-0.6 | scaffold114005 | 6959 | 7234 |
| snap-scaffold5655-processed-gene-0.6 | scaffold5655 | 49042 | 49332 |
| snap-scaffold53979-processed-gene-0.5 | scaffold53979 | 9617 | 9799 |
| evm-scaffold96038-processed-gene-0.1 | scaffold96038 | 71623 | 72027 |
| snap-scaffold120289-processed-gene-0.3 | scaffold120289 | 15738 | 15929 |
| maker-scaffold597-snap-gene-0.30 | scaffold597 | 94782 | 98489 |
| maker-scaffold135148-exonerate_est2genome-gene-0.9 | scaffold135148 | 37858 | 38972 |
| maker-scaffold112101-snap-gene-0.0 | scaffold112101 | 558 | 4634 |
| snap-scaffold17754-processed-gene-0.6 | scaffold17754 | 41594 | 42108 |
| snap-scaffold66720-processed-gene-0.28 | scaffold66720 | 47972 | 48286 |
| snap-scaffold23880-processed-gene-0.19 | scaffold23880 | 145666 | 146250 |
| maker-scaffold154965-snap-gene-0.18 | scaffold154965 | 19696 | 21012 |
| maker-scaffold5618-exonerate_est2genome-gene-0.26 | scaffold5618 | 111062 | 111528 |
| maker-scaffold27133-snap-gene-0.30 | scaffold27133 | 50671 | 52849 |
| snap-scaffold51555-processed-gene-0.24 | scaffold51555 | 110439 | 110771 |
| evm-scaffold89004-processed-gene-0.12 | scaffold89004 | 40733 | 41542 |
| snap_masked-scaffold25641-processed-gene-0.2 | scaffold25641 | 81893 | 82177 |
| snap-scaffold29669-processed-gene-0.4 | scaffold29669 | 70525 | 70887 |
| evm-scaffold112453-processed-gene-0.6 | scaffold112453 | 84131 | 86775 |
| snap-scaffold9956-processed-gene-0.2 | scaffold9956 | 13943 | 15844 |
| snap_masked-scaffold149691-processed-gene-0.6 | scaffold149691 | 13775 | 14008 |
| snap_masked-scaffold15951-processed-gene-0.3 | scaffold15951 | 66902 | 67192 |
| maker-scaffold17870-snap-gene-0.0 | scaffold17870 | 21506 | 22472 |
| snap_masked-scaffold5888-processed-gene-0.0 | scaffold5888 | 18203 | 19313 |
| maker-scaffold96861-exonerate_est2genome-gene-0.48 | scaffold96861 | 91008 | 92647 |
| maker-scaffold75304-snap-gene-0.8 | scaffold75304 | 32568 | 39530 |
| maker-scaffold85799-exonerate_est2genome-gene-0.3 | scaffold85799 | 44744 | 45723 |
| snap_masked-scaffold7926-processed-gene-1.11 | scaffold7926 | 174259 | 174552 |
| maker-scaffold41486-exonerate_est2genome-gene-0.21 | scaffold41486 | 72418 | 72877 |
| snap-scaffold16694-processed-gene-0.28 | scaffold16694 | 128439 | 128801 |
| snap_masked-scaffold27023-processed-gene-0.7 | scaffold27023 | 6270 | 6638 |
| snap-scaffold149077-processed-gene-0.6 | scaffold149077 | 17024 | 17338 |
| snap_masked-scaffold1389-processed-gene-0.12 | scaffold1389 | 187934 | 188233 |
| snap_masked-scaffold37805-processed-gene-0.26 | scaffold37805 | 75715 | 76116 |
| evm-scaffold60124-processed-gene-0.2 | scaffold60124 | 60398 | 60652 |
| snap-scaffold126287-processed-gene-0.21 | scaffold126287 | 44902 | 45132 |
| maker-scaffold15699-exonerate_est2genome-gene-0.11 | scaffold15699 | 34204 | 34719 |
| maker-scaffold131190-exonerate_est2genome-gene-0.9 | scaffold131190 | 6849 | 7378 |
| snap_masked-scaffold383077-processed-gene-0.1 | scaffold383077 | 17378 | 20322 |
| snap-scaffold113751-processed-gene-0.3 | scaffold113751 | 56577 | 56928 |
| snap-scaffold14417-processed-gene-0.23 | scaffold14417 | 35495 | 35719 |
| snap_masked-scaffold143691-processed-gene-0.0 | scaffold143691 | 17167 | 17457 |
| snap-scaffold22024-processed-gene-0.11 | scaffold22024 | 7267 | 7887 |
| snap_masked-scaffold281786-processed-gene-0.0 | scaffold281786 | 22200 | 22643 |
| snap_masked-scaffold49405-processed-gene-0.7 | scaffold49405 | 30954 | 31334 |
| snap_masked-scaffold8695-processed-gene-0.15 | scaffold8695 | 37705 | 38252 |
| snap_masked-scaffold38140-processed-gene-1.16 | scaffold38140 | 150406 | 150717 |
| snap-scaffold59103-processed-gene-0.6 | scaffold59103 | 48886 | 49305 |
| snap_masked-scaffold124521-processed-gene-0.0 | scaffold124521 | 373 | 759 |
| snap-scaffold44955-processed-gene-1.3 | scaffold44955 | 101327 | 101593 |
| maker-scaffold19557-exonerate_est2genome-gene-0.9 | scaffold19557 | 6375 | 7006 |
| snap-scaffold63049-processed-gene-0.6 | scaffold63049 | 6898 | 7185 |
| snap-scaffold12681-processed-gene-0.34 | scaffold12681 | 137021 | 137359 |
| snap-scaffold100333-processed-gene-0.7 | scaffold100333 | 68078 | 68435 |
| snap-scaffold132283-processed-gene-0.9 | scaffold132283 | 14227 | 14598 |
| maker-scaffold23128-exonerate_est2genome-gene-0.0 | scaffold23128 | 55624 | 56855 |
| snap-scaffold49585-processed-gene-0.9 | scaffold49585 | 39805 | 40749 |
| snap_masked-scaffold170217-processed-gene-0.6 | scaffold170217 | 284 | 832 |
| snap_masked-scaffold4828-processed-gene-0.20 | scaffold4828 | 80125 | 80586 |
| snap-scaffold165790-processed-gene-0.12 | scaffold165790 | 21438 | 21743 |
| snap-scaffold72681-processed-gene-0.14 | scaffold72681 | 2228 | 2557 |
| snap-scaffold13217-processed-gene-1.9 | scaffold13217 | 152763 | 153143 |
| snap_masked-scaffold112526-processed-gene-0.1 | scaffold112526 | 5342 | 5608 |
| snap_masked-scaffold126021-processed-gene-0.0 | scaffold126021 | 237 | 743 |
| snap-scaffold26866-processed-gene-0.8 | scaffold26866 | 17201 | 17425 |
| snap-scaffold15883-processed-gene-0.11 | scaffold15883 | 89609 | 89926 |
| snap-scaffold154958-processed-gene-0.7 | scaffold154958 | 44798 | 45049 |
| maker-scaffold85799-exonerate_est2genome-gene-0.0 | scaffold85799 | 2818 | 3674 |
| maker-scaffold49466-exonerate_est2genome-gene-0.1 | scaffold49466 | 3277 | 4209 |
| snap_masked-scaffold70663-processed-gene-0.1 | scaffold70663 | 15650 | 16044 |
| snap_masked-scaffold161560-processed-gene-0.0 | scaffold161560 | 44177 | 44662 |
| snap_masked-scaffold2950-processed-gene-0.0 | scaffold2950 | 11829 | 12179 |
| snap-scaffold285703-processed-gene-0.0 | scaffold285703 | 87 | 635 |
| maker-scaffold76455-exonerate_est2genome-gene-0.2 | scaffold76455 | 42725 | 43264 |
| snap_masked-scaffold106759-processed-gene-0.11 | scaffold106759 | 12108 | 12389 |
| snap-scaffold129183-processed-gene-0.1 | scaffold129183 | 9039 | 9380 |
| snap-scaffold2393-processed-gene-0.34 | scaffold2393 | 49989 | 50330 |
| snap-scaffold185801-processed-gene-0.10 | scaffold185801 | 126046 | 126426 |
| snap_masked-scaffold68245-processed-gene-0.4 | scaffold68245 | 303 | 719 |
| maker-scaffold270646-exonerate_est2genome-gene-0.0 | scaffold270646 | 2214 | 2653 |
| snap-scaffold315078-processed-gene-0.0 | scaffold315078 | 666 | 1793 |
| maker-scaffold13217-exonerate_est2genome-gene-1.53 | scaffold13217 | 203895 | 204872 |

Importantly, gene ontology analysis was performed to better understand the underlying mechanisms behind our set of variably methylated genes. A significant enrichment on genes with functional characteristics related to GTP-binding proteins (also named G proteins) was observed. G proteins regulating a wide variety of cellular activities, and among others, we detected variably methylated genes playing a role in transcription/translation regulation, response to stress, RNA metabolism, and immune response to pathogens. Together, the functional heterogeneity observed within those 321 variably methylated genes could potentially confer plasticity for the marbled crayfish living under different environmental pressures.

### Example 3

### Context-Dependent Methylation Patterns in Marbled Crayfish Populations

In additional steps, we sought to identify specific context-dependent methylation patterns in our core set of 361 variably methylated genes. To identify tissue-specific methylation differences, we applied a Wilcoxon rank sum test for differential (p<0.05 after Benjamini-Hochberg correction) methylation between hepatopancreas and abdominal muscle. For our largest dataset from a single location (Singlis, N=24) this identified 56 genes that allowed a robust separation of the two tissues in a principal component analysis. When the same approach was applied to the second-largest dataset (Reilingen, N=19), it identified 35 differentially methylated genes (28 overlapping with Singlis) that again allowed a robust separation of the two tissues in a principal component analysis. Tissue-specific methylation differences appeared rather moderate for average gene methylation levels, but more pronounced at the CpG level. Of note, tissue-specific methylation differences were highly stable between different populations. Taken together, these findings suggest the existence of localized tissue-specific methylation patterns in marbled crayfish.

To identify location-specific methylation differences, we applied a Kruskal-Wallis test for differential (p<0.05 after Benjamini-Hochberg correction) methylation between the four locations. For the larger hepatopancreas dataset (N=47), this identified 122 genes that allowed a robust separation of the four locations in a principal component analysis. When the same approach was applied to the smaller abdominal muscle dataset (N=26), it identified 22 differentially methylated genes (21 overlapping with hepatopancreas) that again allowed a robust separation of the four locations in a principal component analysis. Similar to our findings for tissue-specific methylation, location-specific methylation differences appeared moderate for average gene methylation levels, but more pronounced at the CpG level. Also, location-specific methylation differences were highly stable between different locations. These findings suggest the existence of defined location-specific methylation differences among marbled crayfish populations.

### Example 4

### Validation of Context Dependent Methylation Patterns

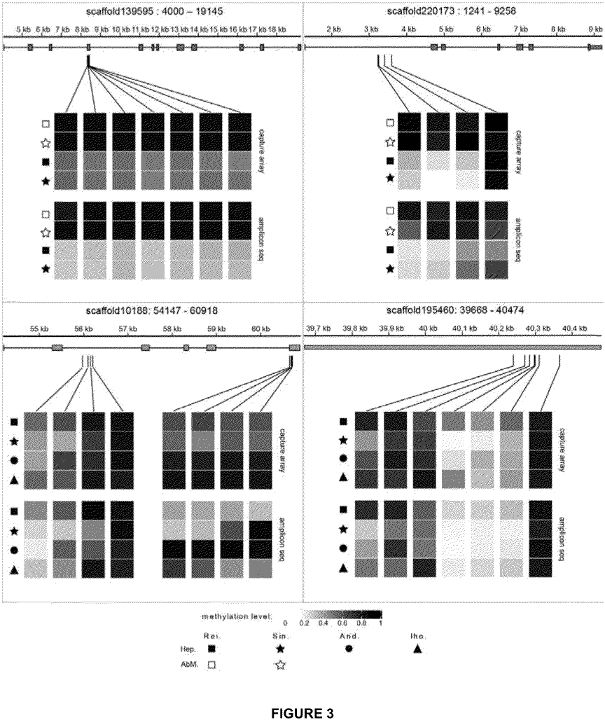

To validate the results for the tissue- and location-specific methylation patterns, markers based on differentially methylated regions (DMRs) within the identified genes, which lead to the separation of the samples, were designed. Both, tissue-specific markers (n=2) and location-specific markers (n=2) were tested with samples from the same two tissues (hepatopancreas and abdominal muscle) and the same four locations (Reilingen, Singlis, Andragnaroa and Ihosy), but from new samples, collected one to two years after the first sampling. The samples were analysed on a PCR based deep sequencing of amplicons. The results confirmed the finding from the capture based subgenome sequencing. With the chosen markers, a separation between the tissues as well as for locations, based on mean methylation ratios per CpG was possible. The mean CpG ratios for the sequenced amplicons were additionally comparable to the mean CpG ratios of the bead-based capture results. Notably, this also confirms that location-specific methylation is stable over time among marbled crayfish populations, resulting in the possibility to define location specific markers to identify the origin of a population and use methylation patterns as a fingerprint for those. These results are shown in FIGS. 2 and 3.

### Materials and Methods

Sampling for bead-based capture assay was carried out in August 2017 for Reilingen, Oktober 2017 for Singlis and as mentioned in Adriantsoa et al., 2019, from October 2017 to March 2018 in Madagascar. Sampling for validation experiment was carried out from March to May 2019 in Germany and Madagascar. Samples were preserved in 100% ethanol and stored in -80° C. until DNA was extracted.

Genomic DNA was isolated and purified from abdominal muscular and hepatopancreas tissue using a Tissue Ruptor (Qiagen), followed by proteinase K digestion and isopropanol precipitation. The quality of isolated genomic DNA was assessed on a 2200 TapeStation (Agilent).

Library preparation was carried out as described in the SureSelectXT Methyl-Seq Target Enrichment System for Illumina Multiplexed Sequencing Protocol, Version D0, July 2015. Quality controls were performed, and sample concentrations were measured on a 2200 TapeStation (Agilent). Multiplexed samples were sequenced on a HiSeqX ten system (Illumina).

Read pairs were quality trimmed and mapped to the 697 genes that showed variable methylation in the whole-genome bisulfite sequencing datasets (Gatzmann et al., 2018) using BSMAP (Xi and Li, 2009). Subsequently, the methylation ratio for each CpG site was calculated using the Python provided with BSMAP. Only those CpG sites that were present in all the samples with a coverage of ≥5x were considered for further analysis. The average methylation level for each gene was calculated only if a gene had at least 5 CpG sites with ≥5x coverage. Furthermore, the genes with following criteria were excluded from subsequent analysis: i) genes that were in the bottom 10% in terms of methylation variance ii) genes with an average methylation level of < 0.1 or > 0.9, and ii) genes with more than 50% Ns in their sequence.

In order to identify tissue-specific methylation differences, a Wilcoxon rank sum test was applied (hepatopancreas vs. abdominal muscle samples from Singlis and Reilingen) and the p-values were corrected for multiple testing using the Benjamini-Hochberg method. Likewise, to identify location-specific methylation differences, a Kuskal-Wallis test was used, and the p-values were corrected for multiple testing using the Benjamini-Hochberg method. Additionally, dmrseq (Korthauer et al., 2018) was used to identify tissue-specific and location-specific differentially methylated regions within the respective genesets.

Genomic DNA was bisulfite converted by using the EZ DNA Methylation-Gold Kit (Zymo Research) following the manufacturer’s instructions. Target regions were PCR amplified using region-specific primers (Tab. 3). PCR products were gel-purified using the QIAquick Gel Extraction Kit (Qiagen). Subsequently, samples were indexed using the Nextera XT index Kit v2 Set A (Illumina). The pooled library was sequenced on a MiSeqV2 system using a paired-end 150 bp nano protocol. Sequencing data was analyzed using BisAMP (BisAMP: A web-based pipeline for targeted RNA cytosine-5 methylation analysis, Bormann F, Tuorto F, Cirzi C, Lyko F, Legrand C.Methods. 2019 Mar 1;156:121-127.)

TABLE 3

Primers for Validation

| | | |
|---|---|---|
| Primer | Sequence | |
| Loc88_R1_fwd | 5′-TTATAATATATTAATGGTTTTGATGA-3′ | SEQ. ID. NO.:1 |
| Loc88_R1_rev | 5′-CACAAAAAACAAAAACTACAAACTC-3′ | SEQ. ID. NO.:2 |
| Loc88_R2_fwd | 5′-ATTATATTTATATTGGATGGATTTAATTTA-3′ | SEQ. ID. NO.:3 |
| Loc88_R2_rev | 5′-AAACAAACATCTTATACAATTCTTCTC-3′ | SEQ. ID. NO.:4 |
| Loc_460_fwd | 5′-GGGTAGATAGAATTATTTTTTTT-3′ | SEQ. ID. NO.:5 |
| Loc_460_rev | 5′-TTTCCTAAAAACCACATTAAAACAC-3′ | SEQ. ID. NO.:6 |
| Tis_595_fwd | 5′-TGGAGATAAGTTAGTTTAATTAGGTTATAT-3′ | SEQ. ID. NO.:7 |
| Tis_595_rev | 5′-AATCATCTTAAAAATTCAAAAAAAA-3′ | SEQ. ID. NO.:8 |
| Tis_173_fwd | 5′-GAATTATTTTATTTGTGATATTTTTTTAAT-3′ | SEQ. ID. NO.:9 |
| Tis_173_rev | 5′-ATTAATCCACATAATATTTCACCAC-3′ | SEQ. ID. NO.:10 |

### Example 5

### Identification of Differentially Methylated CpG Sites in Chicken

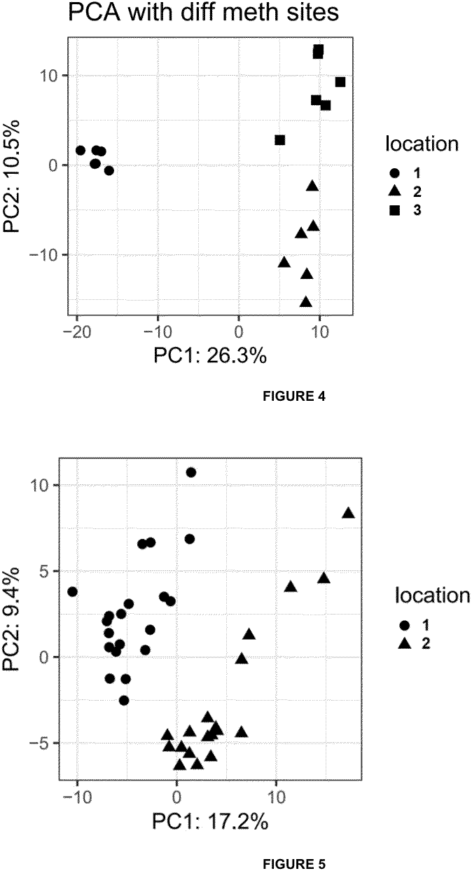

In order to identify differentially methylated CpG sites in the chicken, the function “calculate DiffMeth” from the R package MethylKit was used on the Reduced representation bisulfite sequencing (RRBS) data. 1274 differentially methylated CpGs were identified (p-value < 0.05). Prior to this analysis, the data was filtered for SNPs and a coverage cutoff of minimum 10 per CpG site was applied. The identified differentially methylated CpG sites allowed a robust separation of the three locations in a principle component analysis as shown in FIG. 4.

### Material and Methods

Isolated and purified genomic DNA from breast muscular tissue was provided by different service laboratories in the respective country of sample source. Quality was checked using a 2200 TapeStation (Agilent).

RRBS library preparation was carried out as described in the Zymo-Seq RRBS™ Library Kit Instruction Manual Ver. 1.0.0. Quality controls were performed, and sample concentrations were measured on a 2200 TapeStation (Agilent). Multiplexed samples were sequenced on a HiSeq 4000 system (Illumina).

Reads were quality trimmed using trimmomatic version 0.38 and mapped with BSMAP 2.90 to the Gallus gallus genome assembly version 5.0. Methylation ratios were calculated using a python script (methratio.py) distributed with the BSMAP package. All the CpG sites that were associated with sex chromosomes and the CpG sites that overlapped with SNPs for the Gallus gallus genome were filtered out from the further analysis. Differential methylation analysis was performed using the R package MethylKit (Akalin et al. (2012), Genome Biology, 13(10), R87).

### Example 6

### Identification of Differentially Methylated CpG Sites in Coho Salmon

In order to identify differentially methylated regions in the coho salmon’s RRBS data, the function “calculate DiffMeth” from the R package MethylKit was used. 440 differentially methylated regions were identified (p-value < 0.05, difference in methylation >= 10%). Prior to this analysis, the data was filtered for SNPs and a coverage cutoff of minimum 10 per CpG site was applied. The identified differentially methylated regions allowed a robust separation of the two locations in a principle component analysis as shown in FIG. 5.

### Material and Methods

RRBS data that was published by Le Luyer et al., 2017 was downloaded from the National Center for Biotechnology Information Sequence Read Archive. Reads were mapped with BSMAP 2.90 to Okis_V2 (GCF_002021735.2) and methylation ratios were determined using a python script (methratio.py) distributed with the BSMAP package. All the CpG sites that overlapped with SNPs were filtered out from the further analysis. Differential methylation analysis, with the breeding environment and sex as covariates, was performed using the R package MethylKit (Akalin et al. (2012), Genome Biology, 13(10), R87).

1. A method for the identification of the geographic origin of an individual test subject or of an individual group of test subjects, the method comprising
the comparison of a test methylation profile obtained from genomic material of the individual test subject or of the individual group of test subjects with one or more predetermined reference methylation profiles each being specific for a distinct geographic origin.

2. The method of claim 1, comprising the steps of:
a. determining the methylation status of one or more pre-selected methylation sites within the genomic material contained in a biological sample obtained from the individual test subject, or of the individual group of test subjects;
b. determining from the methylation status determined in (a) a test methylation profile of the individual test subject, or of the individual group of test subjects; and
c. comparing the test methylation profile determined in (b) with one or more predetermined reference methylation profiles, wherein each of the one or more predetermined reference methylation profiles is specific for a distinct geographic origin of subjects or group of subjects which are of the same biological taxon of the individual test subject or individual group of test subjects;
wherein if the test methylation profile is significantly similar to one of the one or more predetermined reference methylation profiles, the individual test subject or the individual group of test subjects has a geographical origin similar to the subjects or group of subjects of the one or more predetermined reference methylation profiles.

3. The method of claim 1, wherein the individual test subject or individual group of test subjects is any biological entity having a DNA genome and DNA genome methylation, preferably the methylation site being a CpG site.

4. The method of claim 1, wherein the individual test subject or individual group of test subjects are selected from a prokaryote, or a eukaryote.

5. The method of claim 2, wherein the one or more pre-selected methylation sites in (a) are methylation sites associated with tissue specific gene expression, preferably wherein the pre-selected methylation sites are associated with gene expression of one distinct tissue.

6. The method of claim 5, wherein the tissue is selected from the group consisting of
(i) metabolic tissue preferably being gut tissue,
(ii) muscular tissue,
(iii) skin or feather tissue, and
(iv) organ tissue, said organ tissue preferably being hepatic and/or pancreatic tissue.

7. The method of claim 1, wherein the individual test subject, or the individual group of test subjects, are animals.

8. The method of claim 1, wherein the distinct geographic origin is a geographic location that is considered to be the habitat, wherein the individual test subject, or individual group of test subjects, were spawned and/or cultured, or at least cultured for a significant time during their lifetime.

9. The method according to claim 1, wherein the one or more pre-selected methylation sites are within the 20% most differentially methylated genes of the genome of the individual test subject, or individual group of test subjects.

10. A method for quality controlling a suspected geographic origin of an individual test subject, or of an individual group of test subjects, the method comprising the steps of
a. determining the methylation status of one or more pre-selected methylation sites within genomic material contained in a biological sample obtained from the individual test subject, or of the individual group of test subjects;
b. determining from the methylation status determined in (a) a test methylation profile of the individual test subject, or of the individual group of test subjects; and
c. comparing the test methylation profile determined in (b) with a predetermined reference methylation profile, wherein the predetermined reference methylation profile is specific for individual subjects, or individual groups of subjects, of the same biological taxon of the individual test subject or individual group of test subjects, and which were obtained from the suspected geographic origin;
wherein if the test methylation profile is significantly similar to the predetermined reference methylation profile, the individual test subject or the individual group of test subjects passes the quality control and the suspected geographical origin is indicated as true geographical origin.

11. A method for assessing one or more environmental parameters of a habitat of an individual test subject, or of an individual group of test subjects, the method comprising the steps of
a. determining the methylation status of one or more pre-selected methylation sites within the genomic material contained in a biological sample obtained from the individual test subject, or of the individual group of test subjects;
b. determining from the methylation status determined in (a) a test methylation profile of the individual test subject, or individual group of test subjects; and
c. comparing the test methylation profile determined in (b) with one or more predetermined reference methylation profiles, wherein the one or more predetermined reference methylation profiles are each specific for individual subjects, or individual groups of subjects, of the same biological taxon of the individual test subject or individual group of test subjects, and which were each obtained from distinct geographic origins; and wherein the distinct geographic origin is distinguished from other distinct geographic origins by one or more environmental parameters;
wherein if the test methylation profile is significantly similar to one of the one or more predetermined reference methylation profiles, the individual test subject or the individual group of test subjects is derived from a geographical origin having similar, or preferably equal, environmental parameters to the geographical origin of the individual test subjects or individual group of test subjects of the one of the one or more predetermined reference methylation profiles.

12. A method for confirming or declining an assumed geographic origin of an individual test subject or of an individual group of test subjects, the method comprising
the comparison of a test methylation profile obtained from genomic material of the individual test subject or of the individual group of test subjects with one or more predetermined reference methylation profiles each being specific for a distinct geographic origin.

13. A method for developing a test system for confirming an assumed geographic origin of an individual test subject or of an individual group of test subjects, the method comprising the steps of:
a. determining the methylation status of one or more methylation sites within genomic material contained in a biological sample obtained from the individual test subject, or of the individual group of test subjects;
b. selecting from the one or more methylation sites a reference panel of methylation sites which is characterized by a specific and distinct differential methylation profile for each of the known geographic origins;
c. obtaining a test system by assigning a reference methylation profile for each of the known geographic origins; and
wherein a comparison of a test methylation profile obtained from a test sample with the reference methylation profiles obtained in (c) allows for confirming the assumed geographic origin of the individual test subject or of the individual group of test subjects from which the test sample was obtained.

14. The method of claim 1, wherein the individual test subject, or the individual group of test subjects is marbled crayfish and/or wherein the distinct geographic origins are geographically distinct waters, these waters preferably being selected from the group consisting of lake(s), river(s) and aquaculture farms.

15. The method of claim 14, wherein the geographically distinct waters are made distinct by one or more environmental parameters selected from the group consisting of pH, water hardness, manganese content, iron content, and aluminum content.

16. The method of any one of claim 14, wherein the method comprises a genome wide methylation analysis or a methylation analysis of a pre-selected panel of methylation sites, the pre-selected panel of methylation sites preferably containing methylation sites within about 500 to 1000, and preferably about 700 genes.

17. The method of claim 16, wherein the panel of methylation sites does not comprise consistently methylated or unmethylated methylation sites.


SEQUENCE LISTING (10 sequences)

SEQ ID NO: 1 - DNA, 26 bp, artificial
  Primer
ttataatatattaatggttttgatga

SEQ ID NO: 2 - DNA, 25 bp, artificial
  Primer
cacaaaaaacaaaaactacaaactc

SEQ ID NO: 3 - DNA, 30 bp, artificial
  Primer
attatatttatattggatggatttaattta

SEQ ID NO: 4 - DNA, 27 bp, artificial
  Primer
aaacaaacatcttatacaattcttctc

SEQ ID NO: 5 - DNA, 23 bp, artificial
  Primer
gggtagatagaattatttttttt

SEQ ID NO: 6 - DNA, 25 bp, artificial
  Primer
tttcctaaaaaccacattaaaacac

SEQ ID NO: 7 - DNA, 30 bp, artificial
  Primer
tggagataagttagtttaattaggttatat

SEQ ID NO: 8 - DNA, 25 bp, artificial
  Primer
aatcatcttaaaaattcaaaaaaaa

SEQ ID NO: 9 - DNA, 30 bp, artificial
  Primer
gaattattttatttgtgatatttttttaat

SEQ ID NO: 10 - DNA, 25 bp, artificial
  Primer
attaatccacataatatttcaccac